(19) United States
(12) Patent Application Publication
(10) Pub. No.: US 2016/0356779 A1
(43) Pub. Date: Dec. 8, 2016

(54) METHODS OF GRADING CARCINOMAS

(71) Applicant: ALBERT EINSTEIN COLLEGE OF MEDICENE, INC., Bronx, NY (US)

(72) Inventors: Peng WU, New Rochelle, NY (US); Sara ROUHANIFARD, Bronx, NY (US); Tianqing ZHENG, Bronx, NY (US)

(73) Assignee: Albert Einstein College of Medicine, Inc., Bronx, NY (US)

(21) Appl. No.: 15/117,472

(22) PCT Filed: Feb. 9, 2015
(86) PCT No.: PCT/US2015/014973

§ 371 Date: Aug. 9, 2016

Related U.S. Application Data
(60) Provisional application No. 61/937,775, filed on Feb. 10, 2014.

(51) Int. Cl. G01N 33/574; A61K 35/15; A61K 35/51

(52) U.S. Cl. CPC G01N 33/57492; A61K 35/51; A61K 35/15; G01N 33/57438; G01N 33/57423; G01N 33/57446; G01N 33/57415; G01N 2400/00

(57) ABSTRACT

Methods for grading carcinomas are provided as well as kits therefor. A method is provided for grading a carcinoma, or suspected carcinoma, comprising obtaining a sample of the carcinoma or suspected carcinoma and contacting the sample with one or more reagents so as to identify LacNAc or Gal.beta.1,4GlcNAc expression on cell surfaces thereof, quantifying the identified LacNAc or Gal.beta.1,4GlcNAc expression, and comparing quantified LacNAc or Gal.beta.1,4GlcNAc expression to one or more predetermined control values, and assigning a grade to the suspected carcinoma based on the quantified LacNAc or Gal.beta.1,4GlcNAc expression being in excess of, or less than, the one or more predetermined control values. Also provided is a method for identifying a sample from a subject as a carcinoma sample. Also provided is a kit for grading carcinoma samples comprising a LacNAc-specific or Gal-1,4GlcNAc-specific glycosylation enzyme and a synthetically-labeled sugar molecule and written instructions for use thereof.

### CROSS-REFERENCE TO RELATED APPLICATIONS

This application claims benefit of U.S. Provisional Application No. 61/937,775, filed Feb. 10, 2014, the contents of which are hereby incorporated by reference.

### STATEMENT OF GOVERNMENT SUPPORT

This invention was made with government support under grant number R01GM093282 awarded by the National Institutes of Health. The government has certain rights in the invention.

### BACKGROUND OF THE INVENTION

The disclosures of all patents, patent application publications and publications referred to in this application, including those cited to by number in parentheses, are hereby incorporated by reference in their entirety into the subject application to more fully describe the art to which the subject invention pertains.

Cells of vertebrate organisms comprise a dense surface layer of glycosylated biomolecules, often called the glycocaylx or the cell's glycome. Cell-surface glycans are key players in intercellular communication, as well as signaling migratory and differentiation pathways during development (1). This explains why stem cells comprise a particular set of glycans on their surface, which change as the cells differentiate (2, 3). On the other hand, dynamic changes in the glycome also accompany the transformation (4). Aberrant glycosylation patterns, including under-expression and over-expression of naturally-occurring glycans, as well as neo-expression of glycans normally restricted to embryonic tissues, can be a hallmark of the tumor phenotype (5, 6). For example, the expression of the Thomsen-Friedenreich antigen (Galβ1-3GalNAcα1-Ser/Thr) is observed in lung, breast and liver cancer tissues, but absent from most normal adult cell types (7, 8).

Despite these exciting discoveries, dynamic changes in glycosylation during development and cancer progression remain poorly characterized. Many key questions have not been unanswered, such as: 1. How do glycosylation patterns change in specific cell types and organs during development? 2. Is there any correlation between glycosylation patterns and cancer progression? 3. Can we manipulate a developmental process by changing surface glycans? The ability to visualize and characterize the dynamic changes in glycosylation in specific cell types and in tissue samples would allow us to address these questions and provide potential clinical tools for disease diagnosis.

Deciphering the molecular function of cell-surface glycans represents a great challenge because of the complexity of glycan biosynthesis and technical hurdles in studying these molecules. Glycans are assembled onto proteins posttranslationally in a step-wise fashion by multiple enzymes. Therefore, they are not amenable to imaging techniques that rely on genetically encoded reporters (e.g. Green Fluorescent Protein—GFP).

Conventional detection of cell and tissue glycans relies almost entirely on lectin- and antibody-based methods. Lectins are particularly useful for enriching N-linked glycoproteins by binding to a conserved pentasaccharide core structure. However, such lectins lack specificity for peripheral glycan epitopes—key mediators of specific cell-surface interactions.

An alternative method for the detection of glycans is the use of bioorthogonal chemical reporters (9, 10). By this method, cells or organisms are first treated with a monosaccharide building block bearing a chemically reactive tag. The modified monosaccharide, when taken up by cells and metabolized, is incorporated into cell-surface glycoconjugates. The bioorthogonal chemical tag then allows covalent conjugation with fluorescent probes for visualization (11), or with affinity probes for enrichment and glycomic analysis (12). This approach has been successfully used for the detection and imaging of mucin O-linked glycans (9), sialylated (9) and fucosylated glycans (9, 13), and cytosolic O-GlcNAcylated proteins (14). However, only monosaccharides are tracked by this strategy, and monosaccharides are often found on many different polysaccharides (15). Higher order glycans, i.e. disaccharides or trisaccharides, of specific composition cannot be uniquely labeled by hijacking their biosynthetic pathways with unnatural monosaccharide building blocks (16, 17). Because peripheral higher order glycans, rather than monosaccharides, encode information for cell-surface receptor recognition to trigger specific downstream signaling (18), there is an urgent need to develop methods for their detection.

The present invention provides an improved method for peripheral higher order glycan detection, and its use in grading carcinomas.

### SUMMARY OF THE INVENTION

A method is provided for grading a carcinoma, or suspected carcinoma, comprising obtaining a sample of the carcinoma or suspected carcinoma and contacting the sample with one or more reagents so as to identify LacNAc or Galβ1,4GlcNAc expression on cell surfaces thereof, quantifying the identified LacNAc or Galβ1,4GlcNAc expression, and comparing quantified LacNAc or Galβ1,4GlcNAc expression to one or more predetermined control values, and assigning a grade to the suspected carcinoma based on the quantified LacNAc or Galβ1,4GlcNAc expression being in excess of, or less than, the one or more predetermined control values.

Also provided is a method for identifying a sample from a subject as a carcinoma sample comprising contacting the sample with one or more reagents so as to identify LacNAc or Galβ1,4GlcNAc expression on cell surfaces thereof, quantifying the identified LacNAc or Galβ1,4GlcNAc expression, and comparing quantified LacNAc or Galβ1,4GlcNAc expression to a predetermined control value, and identifying the sample as a carcinoma wherein the quantified LacNAc or Galβ1,4GlcNAc expression thereof is less than the predetermined control value.

Also provided is a method for identifying a sample from a subject as a carcinoma sample comprising contacting the sample with one or more reagents so as to identify LacNAc or Galβ1,4GlcNAc expression on cell surfaces thereof, quantifying the identified LacNAc or Galβ1,4GlcNAc expression, and comparing quantified LacNAc or Galβ1,4GlcNAc expression to a predetermined control value, and identifying the sample as a carcinoma wherein the quantified LacNAc or Galβ1,4GlcNAc expression thereof is equal to or greater than the predetermined control value.

Also provided is a kit for grading carcinoma samples comprising a LacNAc-specific or Galβ1,4GlcNAc-specific glycosylation enzyme and a synthetically-labeled sugar molecule and written instructions for use thereof.

Also provided is a method for altering cell-surface glycans on a cell, comprising contacting the cell with a LacNAc-specific or Galβ1,4GlcNAc-specific glycosylation enzyme and a sugar molecule so as to permit incorporation of the sugar molecule into the LacNAc or Galβ1,4GlcNAc expressed on a cell surface and thereby alter the cell-surface glycans on a cell on the cell surface.

Also provided is a method of treating a hematologic disease that is treatable with human cord blood cells in a subject, comprising administering to the subject an amount of human cord blood cells that have been contacted with a recombinant LacNAc-specific or Galβ1,4GlcNAc-specific glycosylation enzyme and a fucose, such that the fucose has been incorporated into the LacNAc or Galβ1,4GlcNAc expressed on one or more cell surfaces of the human cord blood cells, effective to treat the hematologic disease.

Also provided is a method of treating a cancer in a subject, comprising administering to the subject an amount of human dendritic cells that have been contacted with a recombinant LacNAc-specific or Galβ1,4GlcNAc-specific glycosylation enzyme and a fucose, such that the fucose has been incorporated into the LacNAc or Galβ1,4GlcNAc expressed on one or more cell surfaces of the human dendritic cells, effective to treat the cancer.

Also provided is a method of treating an autoimmune disease in a subject, comprising administering to the subject an amount of human T cells that have been contacted with a recombinant LacNAc-specific or Galβ1,4GlcNAc-specific glycosylation enzyme and a fucose, such that the fucose has been incorporated into the LacNAc or Galβ1,4GlcNAc expressed on one or more cell surfaces of the human T cells, effective to treat the autoimmune disease.

Also provided is a method of treating a hematologic cancer in a subject, comprising administering to the subject an amount of human T cells that have been obtained from the subject and contacted with a recombinant LacNAc-specific or Galβ1,4GlcNAc-specific glycosylation enzyme and a fucose, such that the fucose has been incorporated into the LacNAc or Galβ1,4GlcNAc expressed on one or more cell surfaces of the human T cells, effective to treat the hematologic cancer.

### BRIEF DESCRIPTION OF THE DRAWINGS

FIG. 1A-1G: a) Chemoenzymatic labeling of LacNAcylated glycans on the cell surface using a two-step approach. b.) 20× photomicrograph of CHoMP LacNAc labeling on a 5 micron paraffin section of intestinal villi from a mouse. LacNAc (green), Dapi (blue) c.) Serial section of mouse intestinal villus labeled with ECA lectin. d.) Serial section of mouse intestinal villus labeled with LEA lectin. e.) CHoMP LacNAc labeling on a serial section without copper catalyst. f.) High magnification image of 5 micron intestinal crypt. g.) LacNAc labeling of single cell suspension of crypt cells.

FIG. 2A-2C: Analysis of CHoMP LacNAc labeling on gastric (2A), lung (2B) and breast adenocarcinoma (2C) TMAs. Samples were normalized to MFI of normal cores. Bar graphs show median MFI with interquartile range.

FIG. 3A-3C: Manipulation of crypt organoid differentiation by in situ glycosylation. The orange colored symbol in 3b represents an isolated small intestinal crypt. 3C: crypts treated with α1,3 FucT and GDP-fucose generated more budding on day 4 compared to the untreated crypts.

### DETAILED DESCRIPTION OF THE INVENTION

[Restatement of all Claims to be Inserted].

A method for grading a carcinoma, or suspected carcinoma, comprising obtaining a sample of the carcinoma or suspected carcinoma and contacting the sample with one or more reagents so as to identify LacNAc or Galβ1,4GlcNAc expression on cell surfaces thereof, quantifying the identified LacNAc or Galβ1,4GlcNAc expression, and comparing quantified LacNAc or Galβ1,4GlcNAc expression to one or more predetermined control values, and assigning a grade to the suspected carcinoma based on the quantified LacNAc or Galβ1,4GlcNAc expression being in excess of, or less than, the one or more predetermined control values.

In an embodiment, the carcinoma, or suspected carcinoma, is a gastric adenocarcinoma and the adenocarcinoma is assigned as a Grade 3 or above carcinoma based on the quantified LacNAc or Galβ1,4GlcNAc expression being in excess of a predetermined control value. In an embodiment, the predetermined control value is estimated or determined from a normal gastric tissue from the same species.

In an embodiment, the carcinoma, or suspected carcinoma, is a lung adenocarcinoma and the adenocarcinoma is assigned as a Grade 1 or above carcinoma based on the quantified LacNAc or Galβ1,4GlcNAc expression being less than a first predetermined control value. In an embodiment, the first predetermined control value is estimated or determined from quantified LacNAc or Galβ1,4GlcNAc expression on a normal lung tissue from the same species. In an embodiment, the carcinoma, or suspected carcinoma, is a lung adenocarcinoma and the adenocarcinoma is assigned as a Grade 2 or above carcinoma based on the quantified LacNAc or Galβ1,4GlcNAc expression being less than a first predetermined control value, but more than a second predetermined control value. In an embodiment, the carcinoma, or suspected carcinoma, is a lung adenocarcinoma and the adenocarcinoma is assigned as a Grade 3 or above carcinoma based on the quantified LacNAc or Galβ1,4GlcNAc expression being less than a first predetermined control value, but more than a third predetermined control value. In an embodiment, the carcinoma, or suspected carcinoma, is a lung adenocarcinoma and the adenocarcinoma is assigned as a Grade 4 or above carcinoma based on the quantified LacNAc or Galβ1,4GlcNAc expression being less than a first predetermined control value, but more than a fourth predetermined control value. In an embodiment, the second predetermined control value is estimated or determined from a grade 1 lung adenocarcinoma from the same species. In an embodiment, the third predetermined control value is estimated or determined from a grade 2 lung adenocarcinoma from the same species. In an embodiment, the fourth predetermined control value is estimated or determined from a grade 3 lung adenocarcinoma from the same species.

In an embodiment, the carcinoma, or suspected carcinoma, is a breast adenocarcinoma and the adenocarcinoma is assigned as a Grade 1-2 carcinoma based on the quantified LacNAc or Galβ1,4GlcNAc expression being less than a first predetermined control value. In an embodiment, the first predetermined control value is estimated or determined from quantified LacNAc or Galβ1,4GlcNAc expression on a normal breast tissue from the same species. In an embodiment, the carcinoma, or suspected carcinoma, is a breast adenocarcinoma and the adenocarcinoma is assigned as a Grade 1-2 or above carcinoma based on the quantified LacNAc or Galβ1,4GlcNAc expression being less than a first predetermined control value, but equal to or more than a second predetermined control value. In an embodiment, the carcinoma, or suspected carcinoma, is a breast adenocarcinoma and the adenocarcinoma is assigned as a Grade 2 carcinoma based on the quantified LacNAc or Galβ1,4GlcNAc expression being less than a first predetermined control value, but equal to or more than a third predetermined control value. In an embodiment, the carcinoma, or suspected carcinoma, is a breast adenocarcinoma and the adenocarcinoma is assigned as a Grade 2-3 or above carcinoma based on the quantified LacNAc or Galβ1,4GlcNAc expression being more than a fourth predetermined control value. In an embodiment, the second predetermined control value is estimated or determined from a grade 1-2 breast adenocarcinoma from the same species. In an embodiment, the third predetermined control value is estimated or determined from a grade 2 breast lung adenocarcinoma from the same species. In an embodiment, the fourth predetermined control value is estimated or determined from a grade 2-3 lung adenocarcinoma from the same species.

A method for identifying a sample from a subject as a carcinoma sample comprising contacting the sample with one or more reagents so as to identify LacNAc or Galβ1,4GlcNAc expression on cell surfaces thereof, quantifying the identified LacNAc or Galβ1,4GlcNAc expression, and comparing quantified LacNAc or Galβ1,4GlcNAc expression to a predetermined control value, and identifying the sample as a carcinoma wherein the quantified LacNAc or Galβ1,4GlcNAc expression thereof is equal to or greater than the predetermined control value.

In an embodiment, the carcinoma, or suspected carcinoma, is a gastric adenocarcinoma and the adenocarcinoma is assigned as a Grade 3 or above carcinoma based on the quantified LacNAc or Galβ1,4GlcNAc expression being equal to or greater than a first predetermined control value. In an embodiment, the carcinoma, or suspected carcinoma, is a breast adenocarcinoma and the adenocarcinoma is assigned as a Grade 3 or above carcinoma based on the quantified LacNAc or Galβ1,4GlcNAc expression being equal to or greater than a first predetermined control value.

As used herein, a predetermined control amount is a value decided or obtained, usually beforehand, as a control. The concept of a control is well-established in the field, and can be determined, in a non-limiting example, empirically from non-afflicted subjects (versus afflicted subjects, including afflicted subjects having different grades of cancers), and may be normalized as desired (in non-limiting examples, for volume, mass, age, location, gender) to negate the effect of one or more variables.

In an embodiment, the sample is a cell-containing sample. In an embodiment, the sample is a tissue sample.

In an embodiment, the predetermined control value(s) is or are cancer-specific predetermined control value(s).

A method is provided for grading a carcinoma, or suspected carcinoma, comprising obtaining a sample of the carcinoma or suspected carcinoma and contacting the sample with one or more reagents so as to identify LacNAc or Galβ1,4GlcNAc expression on cell surfaces thereof, quantifying the identified LacNAc or Galβ1,4GlcNAc expression, and comparing quantified LacNAc or Galβ1,4GlcNAc expression to one or more predetermined control values, and assigning a grade to the suspected carcinoma based on the quantified LacNAc or Galβ1,4GlcNAc expression being in excess of, or less than, the one or more predetermined control values.

Also provided is a method for identifying a sample from a subject as a carcinoma sample comprising contacting the sample with one or more reagents so as to identify LacNAc or Galβ1,4GlcNAc expression on cell surfaces thereof, quantifying the identified LacNAc or Galβ1,4GlcNAc expression, and comparing quantified LacNAc or Galβ1,4GlcNAc expression to a predetermined control value, and identifying the sample as a carcinoma wherein the quantified LacNAc or Galβ1,4GlcNAc expression thereof is less than the predetermined control value.

Also provided is a method for identifying a sample from a subject as a carcinoma sample comprising contacting the sample with one or more reagents so as to identify LacNAc or Galβ1,4GlcNAc expression on cell surfaces thereof, quantifying the identified LacNAc or Galβ1,4GlcNAc expression, and comparing quantified LacNAc or Galβ1,4GlcNAc expression to a predetermined control value, and identifying the sample as a carcinoma wherein the quantified LacNAc or Galβ1,4GlcNAc expression thereof is equal to or greater than the predetermined control value.

In an embodiment of the methods, the carcinoma is an adenocarcinoma. In an embodiment of the methods, the carcinoma is a lung, gastric or breast carcinoma.

In an embodiment of the methods, LacNAc or Galβ1,4GlcNAc expression on a cell surface is identified by a method comprising contacting the cell with a LacNAc-specific or Galβ1,4GlcNAc-specific glycosylation enzyme and a synthetically-labeled sugar molecule so as to permit incorporation of the synthetically-labeled sugar molecule into the LacNAc or Galβ1,4GlcNAc expressed on a cell surface and then detecting the presence of the synthetically-labeled sugar molecule so as to determine LacNAc or Galβ1,4GlcNAc expression on the cell surface.

In an embodiment of the methods, quantifying the identified LacNAc or Galβ1,4GlcNAc expression comprises quantifying the amount of synthetically-labeled sugar molecules so as to thereby quantify the level of expression of LacNAc or Galβ1,4GlcNAc on the cell surface.

In an embodiment of the methods, the LacNAc-specific or Galβ1,4GlcNAc-specific glycosylation enzyme is an isolated α1,3 fucosyltransferase.

In an embodiment of the methods, the LacNAc-specific or Galβ1,4GlcNAc-specific glycosylation enzyme has the same amino acid sequence as, or is, a recombinant H. pylori α1,3 fucosyltransferase.

In an embodiment of the methods, the synthetically-labeled sugar molecule comprises a fucose molecule.

In an embodiment of the methods, the synthetically-labeled sugar molecule comprises a C6-azide-labeled fucose molecule or a C6-alkyne-labeled fucose molecule.

In an embodiment, the methods further comprise tagging the synthetically-labeled sugar molecule with a probe molecule by ligand-assisted CuAAC (azide-alkyne cycloaddition).

In an embodiment, the methods further comprise contacting with BTTES28, BTTPS33 or BTTAA29.

In an embodiment of the methods, the sample is a tissue sample.

In an embodiment of the methods, the sample is obtained from a subject, and wherein the subject is a human subject.

Also provided is a kit for grading carcinoma samples comprising a LacNAc-specific or Galβ1,4GlcNAc-specific glycosylation enzyme and a synthetically-labeled sugar molecule and written instructions for use thereof.

In an embodiment, the kit further comprises CuAAC reagents.

In an embodiment, the kit further comprises BTTES28, BTTPS33 or BTTAA29.

In an embodiment, the LacNAc-specific or Galβ1,4GlcNAc-specific glycosylation enzyme is an isolated α1,3 fucosyltransferase.

In an embodiment, the synthetically-labeled sugar molecule comprises a C6-azide-labeled fucose molecule or a C6-alkyne-labeled fucose molecule.

Also provided is a method for altering cell-surface glycans on a cell, comprising contacting the cell with a LacNAc-specific or Galβ1,4GlcNAc-specific glycosylation enzyme and a sugar molecule so as to permit incorporation of the sugar molecule into the LacNAc or Galβ1,4GlcNAc expressed on a cell surface and thereby alter the cell-surface glycans on a cell on the cell surface.

In an embodiment of the method, the sugar molecule is a fucose. In an embodiment of the method, the fucose is synthetically-labelled. In an embodiment of the method, the fucose is not synthetically-labeled. In an embodiment of the method, the cell is part of a tissue.

In an embodiment, the method further comprises quantifying the effect of altering the cell-surface glycans on a cell on the differentiation of the cell or on the phenotype of the cell.

In an embodiment of the method, the LacNAc-specific or Galβ1,4GlcNAc-specific glycosylation enzyme is an isolated α1,3 fucosyltransferase.

In an embodiment of the method, the LacNAc-specific or Galβ1,4GlcNAc-specific glycosylation enzyme has the same amino acid sequence as, or is, a recombinant H. pylori α1,3 fucosyltransferase.

Also provided is a method of treating a hematologic disease that is treatable with human cord blood cells in a subject, comprising administering to the subject an amount of human cord blood cells that have been contacted with a recombinant LacNAc-specific or Galβ1,4GlcNAc-specific glycosylation enzyme and a fucose, such that the fucose has been incorporated into the LacNAc or Galβ1,4GlcNAc expressed on one or more cell surfaces of the human cord blood cells, effective to treat the hematologic disease.

In an embodiment, the human cord blood cells treated with the recombinant LacNAc-specific or Galβ1,4GlcNAc-specific glycosylation enzyme and a fucose have a higher level of cell-surface fucosylation than untreated human cord blood cells.

In an embodiment, the method further comprises contacting human cord blood cells with a recombinant LacNAc-specific or Galβ1,4GlcNAc-specific glycosylation enzyme and a fucose, such that the fucose is incorporated into the LacNAc or Galβ1,4GlcNAc expressed on one or more cell surfaces of the human cord blood cells, prior to administering the amount of human cord blood cells that have been contacted with a recombinant LacNAc-specific or Galβ1,4GlcNAc-specific glycosylation enzyme and a fucose.

In a embodiment, the hematologic disease is a malignant hematologic disease. In an embodiment, the disease is a non-malignant hematologic disease. In an embodiment, the hematologic disease is a leukemia, an anemia, or a hemoglobinopathy.

In an embodiment, the cells are allogeneic relative to the subject being treated.

Also provided is a method of treating a cancer in a subject, comprising administering to the subject an amount of human dendritic cells that have been contacted with a recombinant LacNAc-specific or Galβ1,4GlcNAc-specific glycosylation enzyme and a fucose, such that the fucose has been incorporated into the LacNAc or Galβ1,4GlcNAc expressed on one or more cell surfaces of the human dendritic cells, effective to treat the cancer. In an embodiment, the cancer is a solid tumor.

Also provided is a method of treating an autoimmune disease in a subject, comprising administering to the subject an amount of human T cells that have been contacted with a recombinant LacNAc-specific or Galβ1,4GlcNAc-specific glycosylation enzyme and a fucose, such that the fucose has been incorporated into the LacNAc or Galβ1,4GlcNAc expressed on one or more cell surfaces of the human T cells, effective to treat the autoimmune disease. In an embodiment, the autoimmune disease is multiple sclerosis.

Also provided is a method of treating a hematologic cancer in a subject, comprising administering to the subject an amount of human T cells that have been obtained from the subject and contacted with a recombinant LacNAc-specific or Galβ1,4GlcNAc-specific glycosylation enzyme and a fucose, such that the fucose has been incorporated into the LacNAc or Galβ1,4GlcNAc expressed on one or more cell surfaces of the human T cells, effective to treat the hematologic cancer. In an embodiment, the hematologic cancer is a B cell cancer. In an embodiment, the B cell cancer is acute lymphoblastic leukemia (ALL).

A method is also provided for treating chimeric antigen receptors or “CAR” T-Cells, that are to be administered to treat a patient, with a recombinant LacNAc-specific or Galβ1,4GlcNAc-specific glycosylation enzyme and a fucose, such that the fucose has been incorporated into the LacNAc or Galβ1,4GlcNAc expressed on one or more cell surfaces of the CAR T cells. Chimeric antigen receptors (CARs)) are engineered receptors, which graft an predetermined specificity onto an immune effector cell. Typically, these receptors are used to graft the specificity of a monoclonal antibody onto a T cell, with transfer of their coding sequence facilitated by retroviral vectors. Such cells are useful in the treatment of cancers. Cell-surface fucosylation by the recited method will improve their performance.

In an embodiment of the methods of treatment, the relevant recited cell type treated with the recombinant LacNAc-specific or Galβ1,4GlcNAc-specific glycosylation enzyme and a fucose have a higher level of cell-surface fucosylation than untreated cells.

In an embodiment of the methods of treatment, the methods further comprise contacting the cells with a recombinant LacNAc-specific or Galβ1,4GlcNAc-specific glycosylation enzyme and a fucose, such that the fucose is incorporated into the LacNAc or Galβ1,4GlcNAc expressed on one or more cell surfaces of the cells, prior to administering the amount of cells.

In an embodiment of the methods of treatment, the fucose is synthetically-labeled. In an embodiment of the methods of treatment, the LacNAc-specific or Galβ1,4GlcNAc-specific glycosylation enzyme is an isolated α1,3 fucosyltransferase. In an embodiment of the methods of treatment, the LacNAc-specific or Galβ1,4GlcNAc-specific glycosylation enzyme is a recombinant H. pylori α1,3 fucosyltransferase. In an embodiment of the methods of treatment, the synthetically-labeled fucose comprises a C6-azide-labeled fucose molecule or a C6-alkyne-labeled fucose molecule. In an embodiment of the methods of treatment, the synthetically-labeled fucose comprises a bioorthogonal chemical reporter at the C-5 position. In an embodiment of the methods of treatment, the methods further comprise tagging the synthetically-labeled fucose with a probe molecule by ligand-assisted CuAAC (azide-alkyne cycloaddition).

In an embodiment of the methods of treatment, the subject is a human subject.

As used herein, “isolated,” in one embodiment, means purified or substantially free of other biological components.

The phrase “and/or” as used herein, with option A and/or option B for example, encompasses the individual embodiments of (i) option A, (ii) option B, and (iii) option A plus option B.

It is understood that wherever embodiments are described herein with the language “comprising,” otherwise analogous embodiments described in terms of “consisting of” and/or “consisting essentially of” are also provided.

Where aspects or embodiments of the invention are described in terms of a Markush group or other grouping of alternatives, the present invention encompasses not only the entire group listed as a whole, but each member of the group subjectly and all possible subgroups of the main group, but also the main group absent one or more of the group members. The present invention also envisages the explicit exclusion of one or more of any of the group members in the claimed invention.

All combinations of the various elements described herein are within the scope of the invention unless otherwise indicated herein or otherwise clearly contradicted by context.

In the event that one or more of the literature and similar materials incorporated by reference herein differs from or contradicts this application, including but not limited to defined terms, term usage, described techniques, or the like, this application controls.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

### Introduction

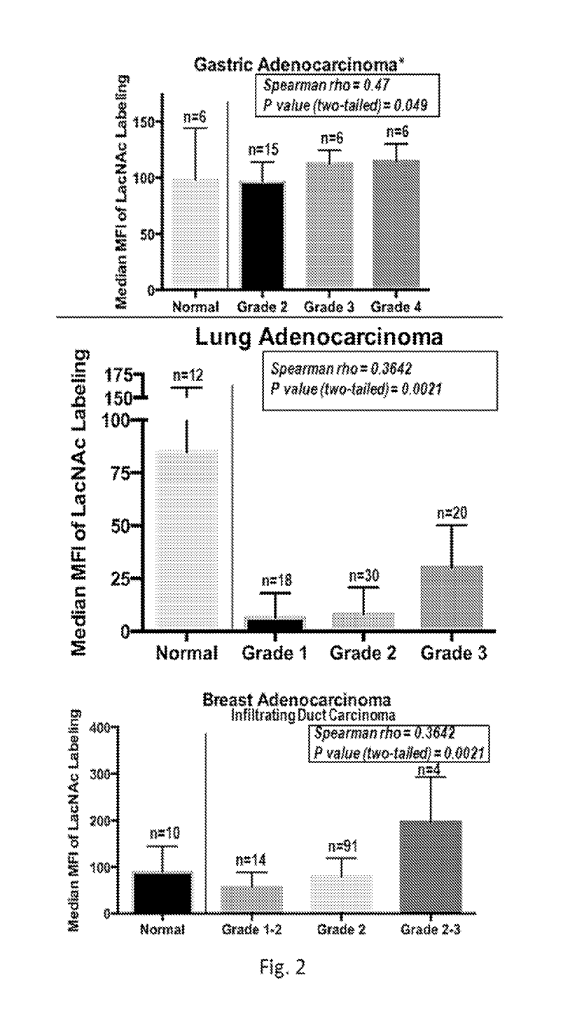

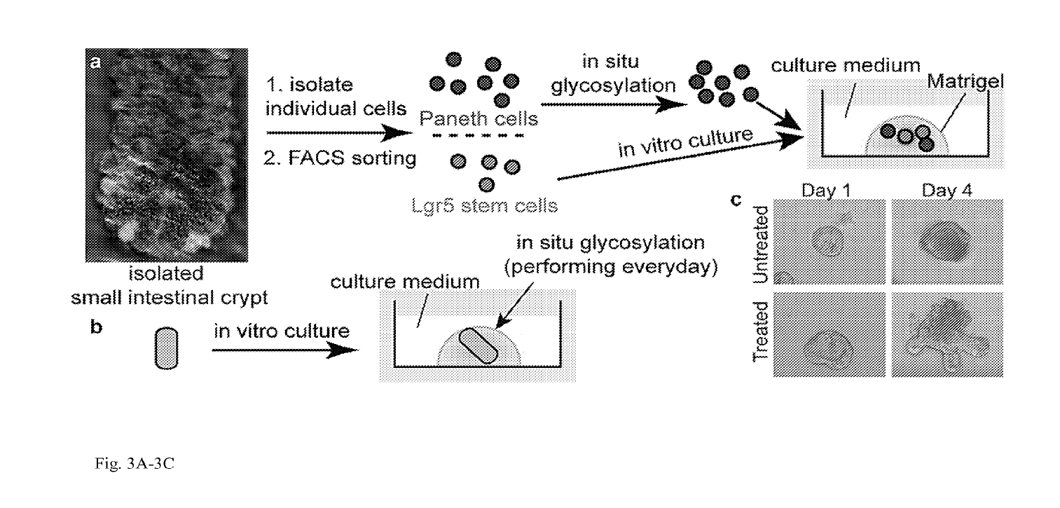

Herein are disclosed chemoenzymatic methods for specific labeling of cell-surface complex glycans with biophysical probes. These methods can be used to image glycomes in living systems and in tissue samples. A two-step labeling process can be employed. First, a glycosyltransferase is used to transfer an azide or alkyne-bearing monosaccharide to target glycans on the cell surface (FIG. 2b). These post-translational modification enzymes are highly specific for their glycan acceptors, but promiscuous towards donor substrates (i.e. nucleotide sugars) (15, 27); thus specific glycan derivatization can be achieved. Subsequently, labeled glycans are reacted with bio-orthogonal probes functionalized in a complementary fashion using, e.g., biocompatible copper(I)-catalyzed azide-alkyne cycloaddition (CuAAC) (FIG. 3a) (28-31), a prototypical example of bio-orthogonal click chemistry (32). Three powerful ligands, BTTES28, BTTPS33 and BTTAA29, dramatically accelerate the kinetics of this reaction, when combined with the in-situ generated copper(I). BTTAA-Cu(I) and BTTPS-Cu(I) represent the fastest and most biocompatible catalytic systems for CuAAC-mediated bioconjugation to date (FIG. 3b).

### Experiments

### Example I

Chemoenzymatic Histology of Membrane Polysaccharides—CHoMP: A Chemoenzymatic Histology Method for Glycan Detection Using ‘Clickable’ Probes: Diseased cells have characteristic, aberrant expression of cell-surface glycans that are under-studied due to a lack of specific methods for labeling polysaccharides on cell surfaces. Recently, this laboratory described a highly specific chemoenzymatic approach for the tagging of N-acetyl lactosamine (LacNAc). Here, this technique is applied towards analyzing expression patterns of LacNAc in tissues or on carcinomas.

Glycans are an important class of biological macromolecules that have diverse roles within the cell and on the cell surface including signal transduction, cell adherence, cell-cell communication, stem cell differentiation and cancer biogenesis. Glycan modifications fluctuate as a consequence of cellular metabolism, developmental state and nutrient availability. They can also fluctuate to induce binding of lectins, and activate signaling cascades. Due to their dynamic nature in response to their environment, glycans can be considered a biomarker for the overall fitness of the cell—in other words, diseased cells have aberrant expression of glycans as compared to healthy cells. The ability to image and characterize the dynamic changes to the glycome would advance our understanding of the detailed roles of glycans in these biological processes.

### Results

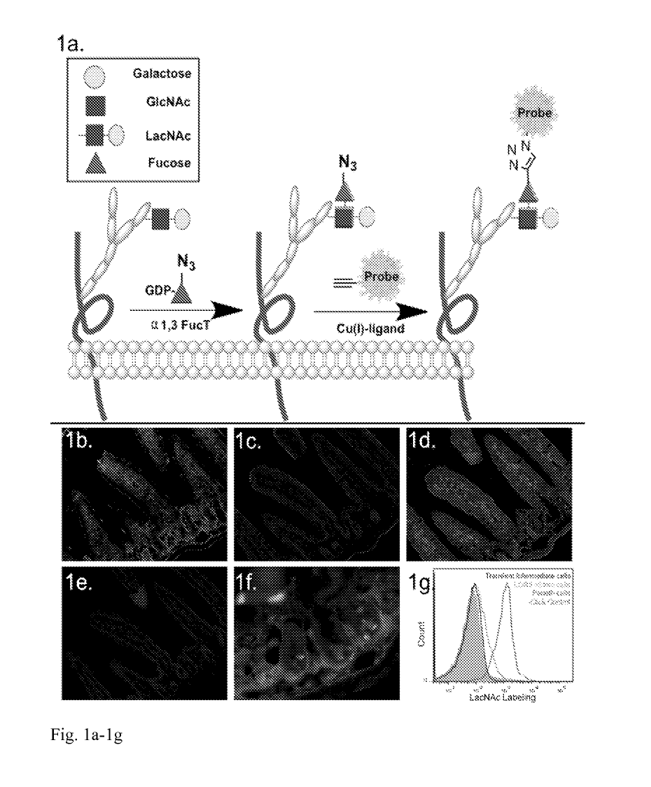

Standard methods for glycan detection involve the use of antibodies and lectins are often limited by their low affinities and poor specificities. The labeling patterns of the two, commercially available lectins commonly used for LacNAc and polyLacNAc labeling on serial, paraffin embedded, mouse intestine sections: Erythrina cristagalli, were compared (ECA; labels LacNAc; FIG. 1c and Lycopersicon esculentum (LEA; labels internal residues of polyLacNAc; FIG. 1d). One would expect to see overlapping patterns from the two lectins, with ECA labeling all LacNAcylated cell types and LEA showing more specific patterns in fewer cell types. Instead, it is seen that ECA and LEA both weakly stain the brush border of the intestinal villi and smooth muscle, however LEA is primarily labeling vasculature in the villi. This clearly demonstrates the shortcomings in regards to specificity of lectin-based histological methods. Furthermore, lectins have well-documented difficulty distinguishing between glucose and galactose residue, as well as their N-acetylated forms respectively. As a result of these limitations, there is relatively little in situ data on the linkage-specific, glycan expression patterns, and histological methods are not utilized in the most effective ways to analyze glycan expression in heterogeneous tissues.

Recently, this laboratory discovered a method that exploits small-molecule probes for the detection of the cell-surface polysaccharide: Type II N-acetyllactosamine (LacNAc, Gal(β1,4)GlcNAc). In an embodiment, this method uses a glycan transfer enzyme (α1,3 fucosyltransferase) to transfer an activated and chemically tagged fucose to the LacNAc residue (FIG. 1a). The chemical tag can subsequently be used for detection using an imaging probe.

LacNAc is a ubiquitously expressed on N-linked glycans as well as some O-linked glycans. LacNAc is generated and its expression controlled through the action of β1,4 galactosyltransferases. The expression can be further controlled through a capping mechanism that attaches sialic acid, fucose or galactose to LacNAc residues. LacNAc is a major substrate for galectins, galactose binding lectins that have been implicated in several biological processes including cell signaling, apoptosis, and cancer, making it an interesting target for the study of biological processes and disease progression.

The chemoenzymatic method for visualizing accessible LacNAc expression patterns on histological samples was adapted in a method (CHoMP), Chemoenzymatic Histology of Membrane Polysaccharides. This technique was then applied towards the two most common applications for histological methods: 1. To identify a function or mechanism by associating staining patterns with known cellular functions, and 2. To use the CHoMP method for LacNAc labeling as a clinical tool to diagnose disease and to help guide therapies.

Following an initial screen of fixed/frozen mouse organs, faint labeling was observed in the spleen and was optimized until a specific pattern emerged. A specific pattern of labeling emerged that was consistent with flow cytometry data showing that naïve T cells have low levels of accessible LacNAc while activated T cells have increased expression. The method was then applied to fixed, paraffin embedded samples to specifically label 8 organs of interest. It was found that the use of surfactant during the enzymatic steps was preferable for labeling.

The specificity of the method was previously reported through the use of cell lines that lack the LacNAc epitope. In addition, no background staining is seen when eliminating the enzymatic step, the addition of tagged sugar, or omitting copper from the “click” reaction (FIG. 1e). This method represents the first example of applying copper catalyzed azide-alkyne cycloaddition (or “click chemistry”) to histological samples in a completely ex vivo method; other reported methods currently require the use of metabolic labeling to introduce the probe.

As a proof of concept, the CHoMP method of labeling LacNAc (FIG. 1b) was then compared to traditional, lectin-based methods for labeling LacNAc and polyLacNAc using ECA (FIG. 1c) and LEA (FIG. 1d). This comparison clearly demonstrates the difference in information gathered from these 3 labeling methods, and the importance of using a method with documented specificity.

Interestingly, a gradient pattern of LacNAc labeling was observed using the CHoMP method on the enterocytes. Progenitor enterocytes have very low expression that increases in a gradient pattern as they age and slough off at the tip of the villus. This phenomenon may account for a previously reported phenomenon that occurs wherein galectin-1 binding to enterocytes triggers apoptosis of aging enterocytes through a caspase mediated mechanism. It was shown that the apoptotic effect is more pronounced at the tip, however the reason why was not discussed. LacNAc is a major substrate for Galectin 1; therefore, the data suggests that the apoptotic effect may be due to increasing levels of LacNAc at the tip of the villus.

Also observed was a strong labeling pattern in the smooth muscle and basement membrane of the intestinal crypt, home to stem cells, Paneth cells and progenitor cells (FIG. 1f). To determine which cell type was responsible for this labeling pattern, we chemoenzymatically labeled LacNAc on a single cell suspension of intestinal crypts from LGR5-GFP mice and co-stained the Paneth cells with UEA lectin and found that Paneth cells were responsible for the pattern of LacNAc labeling (FIG. 1g). Paneth cells maintain stem cell renewal and are responsible for maintaining the stem cell niche in the intestinal crypt. This data, along with an enlarged crypt phenotype observed in a β1,4-galactosyltransferase knockout mouse (this mouse cannot generate LacNAc) together suggest that the glycome of Paneth cells may be responsible for maintaining the stem cell niche in the intestinal crypt.

The histological analysis of human biopsy samples is a powerful diagnostic tool in the medical profession and an important tool for researchers to discover the etiology of various forms of cancer. To diagnose tumors, pathologists begin with an analysis of haemotoxylin and eonsin staining that tells morphological information and helps to determine grade. In some instances, H&E staining is insufficient to get a proper diagnosis, particularly for early detection. In this area, histological techniques may be used. Currently, the primary targets of histology-based cancer diagnostics are protein-based or nucleic acid-based markers (e.g. HER-2 and EGFR) that are detected using direct immunohistochemistry and in situ hybridization, respectively. According to the World Health Organization, lung, stomach, liver, colon and breast cause the most cancer deaths globally per year. To assess the utility of the method towards a general cancer marker, CHoMP was applied to tumor microarrays surveying cancer progression in the stomach, lung and breast. The use of tyramide signal amplification renders this method semiquantitative, however a trend is still possible to observe. A Spearman correlation was calculated and observed a significant trend (p<0.05) for each of the three tumor types as a function of grade, suggesting that LacNAc labeling may be an interesting candidate for diagnosing many types of adenocarcinoma. Of the three tumor types, the lung adenocarcinoma had the most significant trend across grades (p=0.0021), and when compared to normal samples, there was a sharp decrease in accessible LacNAc labeling between the normal samples and the grade 1 tumor samples suggesting a potential for an early detection marker for lung cancer. It has been previously reported that metastatic human lung adenocarcinoma cells display an enhanced expression of α1,3FucT, which could result in LacNAc residues being blocked by fucose residues. Also, overexpression of sialyl Lewis X antigen was found in the sera of lung adenocarcinoma patients—this epitope would also block LacNAc residues from labeling.

In summary, a method for the histological detection of LacNAc epitopes we has been developed using a two-step, enzymatic labeling approach. This method shows greater sensitivity and specificity as compared to traditional lectin based methods for detecting glycans on histological samples. This method was then used to suggest a function for LacNAcylation in the mouse intestine, and also applied toward human tumor microarrays where it displayed a positive correlation with stastistical significance to tumor grade in 3 types of adenocarcinoma (see FIGS. 2A-2C). This method represents the first example of a chemoenzymatic method for detecting sugars on histological samples. It also represents the first example of CuAAc in histological samples without the use of metabolic labeling methods.

### Example II

Manipulation of crypt organoid growth and differentiation by in situ glycosylation: Previous studies by the Clevers lab showed that Paneth cells, a specialized daughter cell derived from intestinal stem cells, constitute the niche for Lgr5 stem cells in the crypt. Paneth cells express cytokines and cofactors that are essential for stem cell maintenance in culture. Single Lgr5-expressing stem cells can be cultured to form long-lived, self-organizing organoids in the absence of non-epithelial niche cells. These organoids retain critical in vivo characteristics such as lineage composition and self-renewal kinetics. Interestingly, co-culturing of sorted stem cells with Paneth cells dramatically improves organoid formation.

Studies herein revealed a unique glycosylation pattern in the crypt, with Paneth cells expressing elevated levels of type II LacNAc than other cell types. Because cell-surface glycans are key mediators of cell-cell communication, it was hypothesized that this unique glycosylation pattern is essential for the dynamic renewing system that maintains the integrity of the intestinal epithelium. By engineering the glycan coating of the crypt, it is possible to develop a powerful method to control and direct this differentiation process.

In situ glycosylation reactions are performed on cells, such as isolated intestinal crypt cells from Lgr5-EGFP-ires-creERT2 mice and the impact of changes in glycan coating on the crypt-villus organoid formation evaluated in vitro (FIG. 3). Fucose or sialic acid residues are added enzymatically to the terminal LacNAc on Paneth cells. Previous studies showed that up-regulation of terminal sialic acid and fucose by over-expressing sialyl- and fucosyltransferases has profound impact on cell-surface receptor signaling and ligand binding. Altered phenotypes are expected to be observed.

A matrigel-based culture system can be used to grow crypt organoids for this study. Isolated crypt cells are sorted from Lgr5-EGFP-ires-creERT2 mice based on EGFP expression (stem cells) and CD24 expression (Paneth cells) (FIG. 3a). Using the sorted Paneth cells, optimal conditions for the α1,3 FucT-mediated cell-surface glycan engineering can be identified to ensure that treated cells remain viable and retain the requisite native phenotype after manipulation. In situ fucosylation reactions are performed on Paneth cells to cap LacNAc epitopes with α1,3 linked fucosides. Stem cells can be seeded alone, or with Paneth cells that are fucosylated, in Matrigel-coated microtiter plates using well-established method. Formation of long-lived organoids is quantified by microscopy every day for 14 days and compared to those formed by co-culturing Lgr5 stem cells with untreated Paneth cells.

In studies herein, it was discovered that the half-life of enzymatically attached fucose residues on the cell surface is approximately 20 hours. In order to maintain high levels of terminal fucosylation on Paneth cells, the experiments can be repeated using intact small intestinal crypts (the orange-colored symbol, FIG. 3b). The Matrigel-based crypt culture system is subjected to in situ fucosylation every 24 hrs. Matrigel permits the penetration of α1,3 FucT and GDP-fucose through the gel to modify LacNAc glycans on the surfaces of the crypt cells. Flow cytometry analysis of a single cell suspension of crypt cells confirmed that LacNAc is almost exclusively present on Paneth cells; therefore, a similar treatment in Matrigel will primarily target Paneth cells. After the fucosylation reaction, the enzyme and nucleotide sugar are removed by washing and replaced with fresh growth medium. Developmental patterns of the treated crypts, such as budding time and frequency, colony formation efficiency, etc. can be compared to untreated controls. Subsequently, cells from the growing organoids can be isolated, stained with cell-type specific markers and the proliferation marker Ki-67 to determine the major cell types that are undergoing proliferation. Apoptosis assays can be used to determine whether in situ fucosylation increases the viability of the aged enterocytes. It was observed that crypts treated with α1,3 FucT and GDP-fucose generated more budding on day 4 compared to the untreated crypts (FIG. 3c), whereas crypts treated with the enzyme alone showed similar growth pattern compared with untreated counterparts.

Recombinant ST6GAL1, ST3GAL4 can be used similarly to add α2,6 and α2,3 linked sialosides to terminal LacNAc on the cell surface.

### Materials and Methods for Examples:

Tissue preparation and processing: Male, 8-12 week old C57BL/6J mice (Jackson Labs) were kept under isoflurane before and during organ perfusion with 50 mL of Tris-HCL buffered Saline (TBS, pH 7.4). Following perfusion with TBS, the mice were perfused with 50 mL of 4% paraformaldehyde (PFA) in TBS. Tissues were harvested and immersed in 4% PFA/TBS for 24 h at 4 degrees C. For frozen sectioning, the tissues were mounted in Tissue-Tek OCT Compound, then frozen in a dry-ice/ethanol bath in a cryomold (Tissue-Tek) then either stored or sectioned at 10 μm slices with a Leica cryostat and mounted ion Superfrost Plus glass slides. For paraffin sectioning, the tissues were paraffin embedded by the Albert Einstein Histology Core Facility, then sectioned on a Leica microtome at 5 μm.

CHoMP Procedure: For paraffin slides, the tissues were deparaffinized and rehydrated according to standard protocols. Note: after deparaffinization and rehydration, the methods for frozen and paraffin sections are the same. A hydrophobic barrier was drawn around the tissue samples on the slides using a PAP pen. Tissues were immersed in 50 mL of TBS+0.1% Tween-20 for 10 minutes in coplin jars. For the enzymatic addition of GDP-fucose-N3, slides were placed in a humidified chamber and 500 μl of enzyme solution (600 μg of α1,3 fucosyltransferase, 350 μM GDP-Fucose-N3, and 5 mM MgCl₂ in TBST) was added to the slides. The slides were incubated for 1 h at 37 degrees. The slides were then washed 3 times in 50 mL of TBST in coplin jars. The slides were again placed in a humidified chamber face-up and an alkyne-tagged, biotin-probe was then ‘clicked’ to the azido-fucose (100 uM biotin-alkyne, 75 uM CuSO₄, 150 uM BTTP ligand, 2.5 mM sodium ascorbate) in TBST for 30 min at RT. Following three washes in TBST, the tissues were blocked for 10 min in 0.3% hydrogen peroxide diluted in TBS in coplin jars at RT, and washed 3× in TBST to remove the H₂O₂. The slides were then places in a humidified chamber and incubated with Neutravidin-HRP (1:100 in TBST) for 1 h at RT, then subsequently washes 3× with TBST in coplin jars. Finally, the slides were placed in a humidified chamber and incubated with TSA-Plus FITC reagent according to manufacturers protocol (1:50 dilution for 10 min, protected from light), then washed 3× for 5 min each in TBST in coplin jars and mounted with Prolong anti-fade gold with Dapi (Invitrogen).

Lectin Staining: Lectin staining was done according to manufacturers protocol using biotinylated ECA and LEA from EY laboratories. Following three washes in TBST, the tissues were blocked for 10 min in 0.3% hydrogen peroxide diluted in TBS in coplin jars at RT, and washed 3× in TBST to remove the H₂O₂. The slides were then places in a humidified chamber and incubated with Neutravidin-HRP (1:100 in TBST) for 1 h at RT, then subsequently washes 3× with TBST in coplin jars. Finally, the slides were placed in a humidified chamber and incubated with TSA-Plus FITC reagent according to manufacturers protocol (1:50 dilution for 10 min, protected from light), then washed 3× for 5 min each in TBST in coplin jars and mounted with Prolong anti-fade gold with Dapi (Invitrogen).

Cytokeratin counterstaining for TMAs: CHoMP labeling was performed according to protocol above without mounting. Slides were subjected to antigen retrieval after CHoMP labeling with Vector unmasking solution in a coplin jar heated to 95 degrees for 20 min. Coplin jar was removed and allowed to cool at RT for 30 min. Slides were laid flat in a humidified chamber and blocked with 5% horse serum with 2% BSA in TBST for 30 min at RT. Slides were then incubated with mouse anti-cytokeratin (Sigma) diluted in serum block (1:100) for 1 h at RT. Slides were rinsed 3× in TBST in a coplin jar, then laid flat in a humidified chamber. The slides were incubated with goat anti-mouse IgG Alexa 555 diluted in serum block 1:250 for 1 h at RT. The slides were then washed 3× for 5 min each in TBST in coplin jars and mounted with Prolong anti-fade gold with Dapi (Invitrogen).

Image analysis: Photomicrograms were acquired on Zeiss Axioobserver digital light microscope and processed using ImageJ software.

Statistical analysis: For TMA analysis, LacNAc labeling was quantified in epithelial cell regions (cytokeratin positive regions), and normalized to the average mean fluorescence intensity (MFI) of the normal cores. Nonparametric analysis was performed on data sets and Spearman correlation was calculated using Prism 6 software.

TMA Description: TMAs (US Biomax, Rockville, Md.) for human adenocarcinomas of the stomach, lung and breast were examined.

Crypt Cell Isolation: Adapted from Barker et al. 2007, Merlos-Suarez et al. 2011, and Sato & Clevers. Briefly, small intestine was excised from Lgr5-GFP-IRES-CreER mouse and flushed with cold PBS, the cut longitudinally. Villi were scraped using a glass coverslip, and cut into 2-4 mm pieces with scissors, then washed 10× with cold PBS. Incubate villi in 2 mM EDTA/PBS at 4 degrees on a roller for 45 min, remove the EDTA and wash/shake 4× with cold PBS and collect 4 fractions. Check under microscope for fractions containing crypts. Pass crypt fractions through a 70 um strainer into a BSA-coated collection tube, spin down and disgard supernatant. Resuspend in 2 mL single-cell dissociation media (DMEM/F12, 2 mM Glutamax, 10 mM Hepes, 1:100 N2, 1:50 B27, and 10 uM Y-27632) and incubate at 37 degrees for 45 min. After washes, the cells are ready for staining and flow cytometry analysis.

Flow cytometry: LacNAc labeling was performed on single cell suspension of crypt cells from Lgr5-GFP mice using alexa Fluor 647. Cells were also stained with a Paneth cell marker, Rhodamine UEA-I (Vector) for 30 min at RT according to manufacturers protocol. Cell suspension was processed through (name of flow cytometer) and analyzed using FlowJo software.

### Example III

The recombinant, isolated H. pylori alfa 1,3 fucosyltransferase can be used to effect in situ cell surface fucosylation of human cord blood cells. The resultant modififed cell product will enhance engraftment efficiency and success level in transplant into a subject's bone marrow for treatment purposes. Umbilical cord blood (UCB) has been used successfully as a source of hematopoietic stem cells (HSCs) for allogeneic transplantation in children and adults in the treatment of hematologic diseases (see, for example, Ren Z, Jiang Y (2013) Umbilical Cord Blood Hematopoietic Stem Cell Expansion Ex Vivo. J Blood Disorders Transf S3: 004. doi:10.4172/2155-9864.53-004, for examples of disroderds that can be treated by the invention and methods of administration (hereby incorporated by reference)). The cell-surface fucosylated cord blood cell product has improved performance over untreated.

Similarly, recombinant, isolated H. pylori alfa 1,3 fucosyltransferase can be used to effect in situ cell surface fucosylation of human dendritic cells to enhance entry into inflamed tissues. This is especially useful in cancers, for example solid tumor cancers.

Recombinant, isolated H. pylori alfa 1,3 fucosyltransferase can be used to effect in situ cell surface fucosylation of T-cells which results in improved homing to sites of inflammation. This strategy can be used in autoimmune diseases amenable to T-cell based therapies, such as Multiple Sclerosis (MS). It can also be used in CAR T-Cell Therapy to Engineer Patients' Immune Cells to Treat Their Cancers such as acute lymphoblastic leukemia (ALL), and other B-cell cancers.

### REFERENCES

1. Ohtsubo K, Marth J D. Glycosylation in cellular mechanisms of health and disease. Cell. 2006; 126(5):855-867. doi: 10.1016/j.cell.2006.08.019. PubMed PMID: 16959566.
2. An H J, Gip P, Kim J, Wu S, Park K W, McVaugh C T, et al. Extensive determination of glycan heterogeneity reveals an unusual abundance of high mannose glycans in enriched plasma membranes of human embryonic stem cells. Molecular & cellular proteomics: MCP. 2012; 11(4):M111 010660. doi: 10.1074/mcp.M111.010660. PubMed PMID: 22147732; PubMed Central PMCID: PMC3322563.
3. Nairn A V, Aoki K, dela Rosa M, Porterfield M, Lim J M, Kulik M, et al. Regulation of glycan structures in murine embryonic stem cells: combined transcript profiling of glycan-related genes and glycan structural analysis. J Biol Chem. 2012; 287(45):37835-37856. doi: 10.1074/jbc.M112.405233. PubMed PMID: 22988249; PubMed Central PMCID: PMC3488057.
4. Haltiwanger R S, Lowe J B. Role of glycosylation in development. Annu Rev Biochem. 2004; 73:491-537. PubMed PMID: 15189151.
5. Guglietta A, Sullivan P B. Clinical applications of epidermal growth factor. Eur J Gastroenterol Hepatol. 1995; 7(10):945-950. PubMed PMID: 8590139.
6. Hahm K B, Im Y H, Parks T W, Park S H, Markowitz S, Jung H Y, et al. Loss of transforming growth factor beta signalling in the intestine contributes to tissue injury in inflammatory bowel disease. Gut. 2001; 49(2):190-198. PubMed PMID: 11454793; PubMed Central PMCID: PMC1728415.
7. Lin W M, Karsten U, Goletz S, Cheng R C, Cao Y. Expression of CD176 (Thomsen-Friedenreich antigen) on lung, breast and liver cancer-initiating cells. Int J Exp Pathol. 2011; 92(2):97-105. doi: 10.1111/j.1365-2613.2010.00747.x. PubMed PMID: 21070402; PubMed Central PMCID: PMC3081512.
8. Hanisch F G, Baldus S E. The Thomsen-Friedenreich (TF) antigen: a critical review on the structural, biosynthetic and histochemical aspects of a pancarcinoma-associated antigen. Histol Histopathol. 1997; 12(1):263-281. PubMed PMID: 9046061.
9. Laughlin S T, Bertozzi C R. Imaging the glycome. Proc Natl Acad Sci USA. 2009; 106(1):12-17. PubMed PMID: 19104067.
10. Rouhanifard S H, Nordstrom L U, Zheng T, Wu P. Chemical probing of glycans in cells and organisms. Chem Soc Rev. 2013; 42(10):4284-4296. doi: 10.1039/c2cs35416k. PubMed PMID: 23257905; PubMed Central PMCID: PMC3641795.
11. Earl L A, Bi S, Baum L G. N- and O-glycans modulate galectin-1 binding, CD45 signaling, and T cell death. J Biol Chem. 2010; 285(4):2232-2244. doi: 10.1074/jbc.M109.066191. PubMed PMID: 19920154; PubMed Central PMCID: PMC2807281.
12. Hanson S R, Hsu T L, Weerapana E, Kishikawa K, Simon G M, Cravatt B F, et al. Tailored glycoproteomics and glycan site mapping using saccharide-selective bioorthogonal probes. J Am Chem Soc. 2007; 129(23):7266-7267. PubMed PMID: 17506567.
13. Hsu T L, Hanson S R, Kishikawa K, Wang S K, Sawa M, Wong C H. Alkynyl sugar analogs for the labeling and visualization of glycoconjugates in cells. Proc Natl Acad Sci USA. 2007; 104(8):2614-2619. PubMed PMID: 17296930.
14. Vocadlo D J, Hang H C, Kim E J, Hanover J A, Bertozzi C R. A chemical approach for identifying O-GlcNAc-modified proteins in cells. Proc Natl Acad Sci USA. 2003; 100(16):9116-9121. PubMed PMID: 12874386.
15. Varki A, Cummings R D, Esko J D, Freeze H H, Stanley P, Bertozzi C R, et al. Eseentials of Glycobiology. 2 ed. New York: Cold Spring Harbor; 2008.
16. Zheng T, Jiang H, Gros M, Soriano Del Amo D, Sundaram S, Lauvau G, et al. Tracking N-acetyllactosamine on cell-surface glycans in vivo. Angew Chem Int Ed. 2011; 50(18):4113-4118. PubMed PMID: 21472942.
17. Chaubard J L, Krishnamurthy C, Yi W, Smith D F, Hsieh-Wilson L C. Chemoenzymatic probes for detecting and imaging fucose-alpha(1-2)-galactose glycan biomarkers. J Am Chem Soc. 2012; 134(10):4489-4492. doi: 10.1021/ja211312u. PubMed PMID: 22339094; PubMed Central PMCID: PMC3303202.
18. Gringhuis S I, den Dunnen J, Litjens M, van der Vlist M, Geijtenbeek T B. Carbohydrate-specific signaling through the D C-SIGN signalosome tailors immunity to Mycobacterium tuberculosis, HIV-1 and Helicobacter pylori. Nature immunology. 2009; 10(10):1081-1088. doi: 10.1038/ni.1778. PubMed PMID: 19718030.

1. A method for grading a carcinoma, or a suspected carcinoma, comprising obtaining a sample of the carcinoma or suspected carcinoma and contacting the sample with one or more reagents so as to identify LacNAc or Galβ1,4GlcNAc expression on cell surfaces thereof, quantifying the identified LacNAc or Galβ1,4GlcNAc expression, and comparing quantified LacNAc or Galβ1,4GlcNAc expression to one or more predetermined control values, and assigning a grade to the suspected carcinoma based on the quantified LacNAc or Galβ1,4GlcNAc expression being in excess of, or less than, the one or more predetermined control values.

2-3. (canceled)

4. The method of claim 1, wherein the carcinoma is an adenocarcinoma.

5. The method of claim 1, wherein the carcinoma is a lung, gastric or breast carcinoma.

6. The method of claim 1, wherein LacNAc or Galβ1,4GlcNAc expression on a cell surface is identified by a method comprising contacting the cell with a LacNAc-specific or Galβ1,4GlcNAc-specific glycosylation enzyme and a synthetically-labeled sugar molecule so as to permit incorporation of the synthetically-labeled sugar molecule into the LacNAc or Galβ1,4GlcNAc expressed on a cell surface, and then detecting the presence of the synthetically-labeled sugar molecule so as to determine LacNAc or Galβ1,4GlcNAc expression on the cell surface.

7. The method of claim 6, wherein quantifying the identified LacNAc or Galβ1,4GlcNAc expression comprises quantifying the amount of synthetically-labeled sugar molecules so as to thereby quantify the level of expression of LacNAc or Galβ1,4GlcNAc on the cell surface.

8. The method of claim 6, wherein the LacNAc-specific or Galβ1,4GlcNAc-specific glycosylation enzyme is an isolated α1,3 fucosyltransferase.

9. The method of claim 6, wherein the LacNAc-specific or Galβ1,4GlcNAc-specific glycosylation enzyme has the same amino acid sequence as, or is, a recombinant H. pylori α1,3 fucosyltransferase.

10. The method of claim 6, wherein the synthetically-labeled sugar molecule comprises a fucose molecule.

11. The method of claim 6, wherein the synthetically-labeled sugar molecule comprises a C6-azide-labeled fucose molecule or a C6-alkyne-labeled fucose molecule.

12. The method of claim 6, further comprising tagging the synthetically-labeled sugar molecule with a probe molecule by ligand-assisted CuAAC (azide-alkyne cycloaddition).

13. The method of claim 12, further comprising contacting with BTTES28, BTTPS33 or BTTAA29.

14. The method of claim 1, wherein the sample is a tissue sample.

15. The method of claim 1, wherein the sample is obtained from a subject, and wherein the subject is a human subject.

16-28. (canceled)

29. A method of treating a hematologic disease that is treatable with human cord blood cells in a subject, comprising administering to the subject an amount of human cord blood cells that have been contacted with a recombinant LacNAc-specific or Galβ1,4GlcNAc-specific glycosylation enzyme and a fucose, such that the fucose has been incorporated into the LacNAc or Galβ1,4GlcNAc expressed on one or more cell surfaces of the human cord blood cells, effective to treat the hematologic disease.

30. The method of claim 29, wherein the human cord blood cells treated with the recombinant LacNAc-specific or Galβ1,4GlcNAc-specific glycosylation enzyme and a fucose have a higher level of cell-surface fucosylation than untreated human cord blood cells.

31. The method of claim 29, further comprising contacting human cord blood cells with a recombinant LacNAc-specific or Galβ1,4GlcNAc-specific glycosylation enzyme and a fucose, such that the fucose is incorporated into the LacNAc or Galβ1,4GlcNAc expressed on one or more cell surfaces of the human cord blood cells, prior to administering the amount of human cord blood cells that have been contacted with a recombinant LacNAc-specific or Galβ1,4GlcNAc-specific glycosylation enzyme and a fucose.

32. A method of treating a cancer in a subject, comprising administering to the subject an amount of human dendritic cells that have been contacted with a recombinant LacNAc-specific or Galβ1,4GlcNAc-specific glycosylation enzyme and a fucose, such that the fucose has been incorporated into the LacNAc or Galβ1,4GlcNAc expressed on one or more cell surfaces of the human dendritic cells, effective to treat the cancer.

33-36. (canceled)

37. The method of claim 29, wherein the fucose is synthetically-labeled.

38-43. (canceled)
